(19) United States
(12) Patent Application Publication
(10) Pub. No.: US 2026/0060652 A1
(43) Pub. Date: Mar. 5, 2026

(54) MEDICAL DEVICE AND METHOD OF MANUFACTURING MEDICAL DEVICE

(71) Applicant: ASAHI INTECC CO., LTD., Seto-shi (JP)

(72) Inventor: Hirotomo SHIMIZU, Seto-shi (JP)

(73) Assignee: ASAHI INTECC CO., LTD., Seto-shi (JP)

(21) Appl. No.: 19/383,788

(22) Filed: Nov. 10, 2025

Related U.S. Application Data
(63) Continuation of application No. PCT/JP2023/018775, filed on May 19, 2023.

(51) Int. Cl. A61B 8/00; A61B 8/12; A61M 25/00; A61M 25/01; B05B 15/50

(52) U.S. Cl. CPC A61B 8/445; A61B 8/12; A61M 25/0009; A61M 25/0045; A61M 25/0108; B05B 15/50

(57) ABSTRACT

A medical device includes a first tube having a first lumen and a second tube having a second lumen, wherein the first tube is coupled to the second tube by a coating agent along at least one coupling portion in a longitudinal direction of the medical device.

### CROSS-REFERENCE TO RELATED APPLICATION

The present application is a bypass continuation of International Application No. PCT/JP2023/018775 filed on May 19, 2023, the entire contents of which being incorporated herein by reference.

### TECHNICAL FIELD

The disclosed embodiments relate to a medical device.

### BACKGROUND ART

There are known medical devices that acquire ultrasonic images. For example, Patent Literature 1 discloses a medical device including a transducer unit that transmits and receives ultrasonic waves to and from a living body tissue, an image lumen that allows the transducer unit and a drive shaft to move, and a guide wire lumen that allows a guide wire to be inserted therethrough. Hereinafter, an ultrasonic image acquisition mechanism including the drive shaft and the transducer unit is also referred to as a “sensor”. By performing a percutaneous procedure using such a medical device, the safety and efficiency of the procedure can be improved.

### CITATION LIST

### Patent Literature

Patent Literature 1: JP 2017-153621 A

### SUMMARY

### Technical Problems

Here, in order to make it possible to operate a therapeutic device (e.g., a plasma guide wire or a penetration guide wire) under sensor observation in the above-described medical device, a plurality of lumens is provided by combining a plurality of tubes. In this regard, in the device described in Patent Literature 1, a first tubular body and a second tubular body are coupled to each other by heat fusion or adhesion. However, when the first and second tubular bodies made of a resin material are thermally fused, there is a disadvantage in that the first and second tubular bodies are deformed due to heat. In addition, when the first and second tubular bodies adhere to each other, there is a disadvantage (e.g., a decrease in the flexibility and the operability and a decrease in the acoustic characteristics) caused by curing of an adhesive.

The above disadvantages are not limited to a medical device having a sensor built therein and also are common to a medical device that is configured to allow a sensor to be inserted thereto and is used in combination with a sensor. In addition, the above disadvantages are not limited to a vascular system and are common to medical devices inserted into various organs in the human body, such as a lymphatic system, a biliary system, a urinary system, an airway system, a digestive system, a secretory gland, and a reproductive organ.

The disclosed embodiments have been made to solve at least a part of the above-described disadvantages and are directed to avoiding disadvantages that may occur due to the use of an adhesive while suppressing deformation of a tube in a medical device including a plurality of tubes.

### Solutions to Problems

The disclosed embodiments have been made to solve at least a part of the above-described and other disadvantages and can be realized as the following aspects.

(1) According to an aspect of the disclosed embodiments, a medical device is provided. The medical device includes a first tube having a first lumen and a second tube that has a second lumen and is coupled to the first tube with a coating agent in a part of a longitudinal direction of the medical device.

It should be noted that the disclosed embodiments can be realized in various aspects, and for example, can be realized in aspects such as a medical device, a medical tube, a catheter, and manufacturing methods thereof.

### BRIEF DESCRIPTION OF DRAWINGS

FIG. 1 is an explanatory view illustrating a configuration of a medical device.

FIG. 2 is an explanatory view illustrating a configuration of the medical device.

FIGS. 3A to 3E are transverse sectional views of a catheter.

FIG. 4 is a diagram illustrating a method of using the catheter.

FIG. 5 is a diagram illustrating a method of using the catheter.

FIG. 6 is an enlarged view of a part of the catheter on a distal end side.

FIG. 7 is a transverse sectional view of the catheter taken along the line F-F in FIG. 6.

FIG. 8 is a transverse sectional view of a catheter according to a second embodiment.

FIG. 9 is a transverse sectional view of a catheter according to a third embodiment.

FIG. 10 is an enlarged view of a part of a catheter on the distal end side according to a fourth embodiment.

FIG. 11 is an enlarged view of a part of a catheter on the distal end side according to a fifth embodiment.

FIG. 12 is an enlarged view of a part of a catheter on the distal end side according to a sixth embodiment.

### DETAILED DESCRIPTION

### First Embodiment

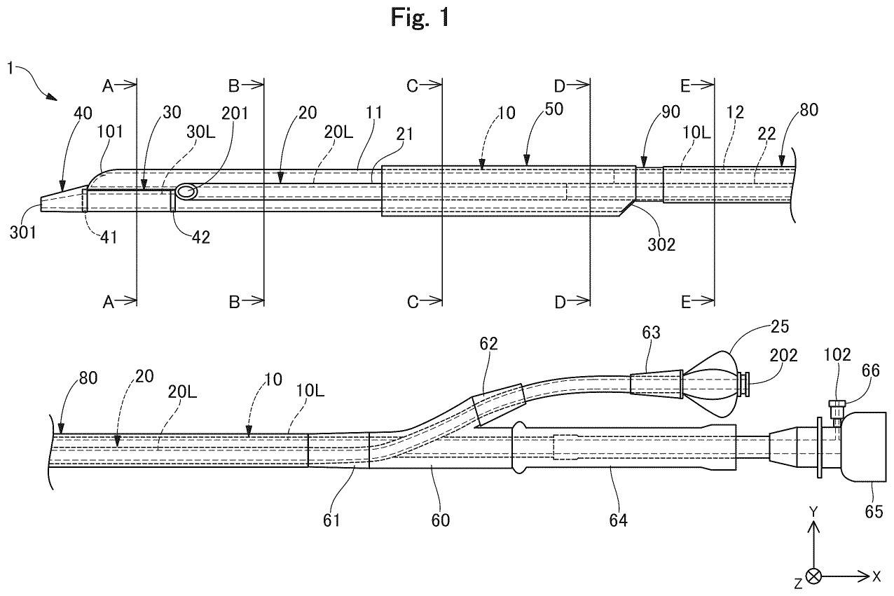

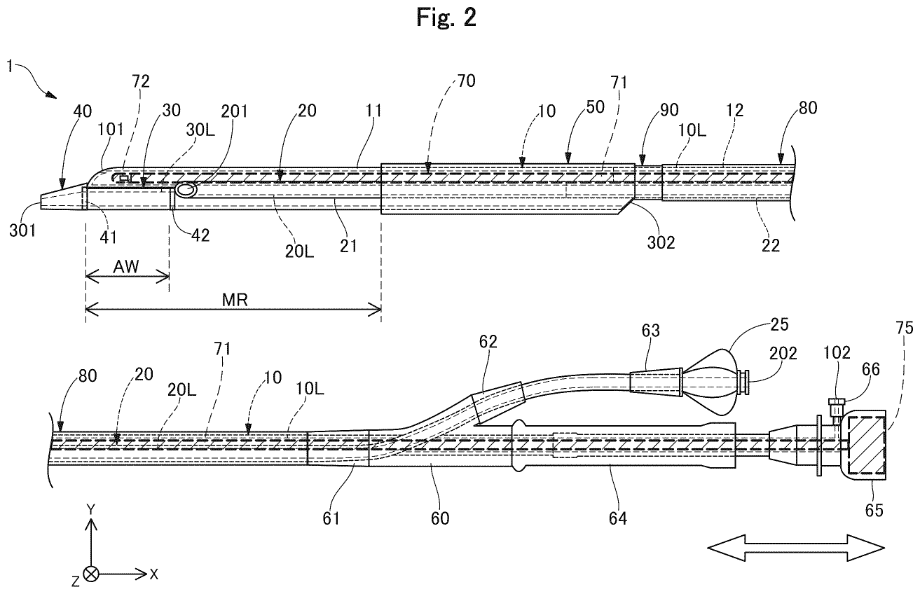

FIGS. 1 and 2 are explanatory views illustrating a configuration of a medical device 1. The medical device 1 according to the present embodiment is, for example, a catheter used for treating a lesion in a living body lumen, such as a CTO occurring in a blood vessel. Hereinafter, the medical device 1 is also referred to as the “catheter 1”. As illustrated in FIGS. 1 and 2, the catheter 1 includes a sensor tube 10, an OTW (Over The Wire) tube 20, an RX (Rapid Exchange) tube 30, a distal tip 40, a first marker 41, a second marker 42, a first outer tube 50, a branching connector 60, a first reinforcing member 61 to a third reinforcing member 63, a cylindrical member 64, a connector 65, a connector 25, a sensor 70, a second outer tube 80, and a heat-shrinkable tube 90. The sensor tube 10 is also referred to as a “medical device” or a “medical tube”. The OTW tube 20 is also referred to as a “medical device”or a “medical tube”.

In FIG. 1, the sensor 70 is not illustrated in order to explain the configurations of a tube and a lumen in the tube. In FIG. 2, the sensor 70 built in a sensor lumen 10L in the sensor tube 10 is indicated by a dashed line and hatched with oblique lines.

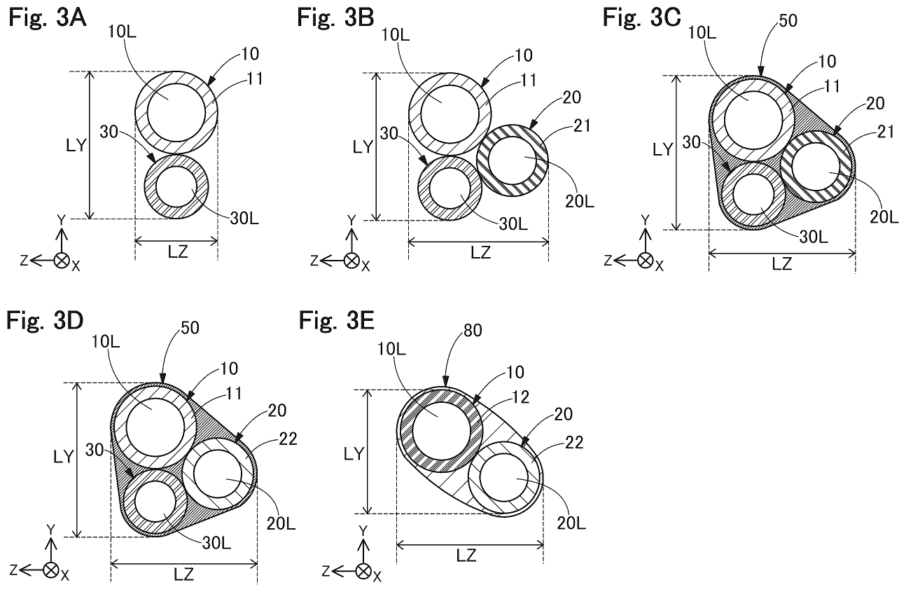

In FIGS. 1 and 2, for convenience of descriptions, the relative ratio of the size of each constituent member includes a portion different from the actual one. Further, a part of each constituent member includes an exaggerated portion. FIGS. 1 and 2 illustrate the XYZ axes orthogonal to each other. The X-axis corresponds to the longitudinal direction of the catheter 1, the Y-axis corresponds to the height direction of the catheter 1, and the Z-axis corresponds to the width direction of the catheter 1. The left side (−X-axis direction) of FIGS. 1 and 2 is referred to as a “distal end side” of the catheter 1 and each constituent member, and the right side (+X-axis direction) of FIGS. 1 and 2 is referred to as a “proximal end side”of the catheter 1 and each constituent member. Further, among both ends of the catheter 1 and each constituent member in the longitudinal direction (X-axis direction), one end located on the distal end side is referred to as a “distal end”, and the other end located on the proximal end side is referred to as a “proximal end”. The distal end and the vicinity thereof are referred to as a “distal end portion”, and the proximal end and the vicinity thereof are referred to as a “proximal end portion”. The distal end side is inserted into the living body, and the proximal end side is operated by a professional such as a doctor. These points are common to FIGS. 3A to 3E and subsequent drawings.

FIGS. 3A to 3E are transverse sectional views of the catheter 1. FIG. 3A illustrates a transverse section of the catheter 1 taken along the line A-A in FIG. 1. FIG. 3B illustrates a transverse section of the catheter 1 taken along the line B-B in FIG. 1. FIG. 3C illustrates a transverse section of the catheter 1 taken along the line C-C in FIG. 1. FIG. 3D illustrates a transverse section of the catheter 1 taken along the line D-D in FIG. 1. FIG. 3E illustrates a transverse section of the catheter 1 taken along the line E-E in FIG. 1. The configuration of the catheter 1 will be described below with reference to FIGS. 1 to 3E.

The sensor tube 10 is a hollow cylindrical member (tubular body) having an elongated outer shape. The sensor tube 10 extends linearly along the longitudinal direction (X-axis direction) of the catheter 1 in parallel with the OTW tube 20 and the RX tube 30. The sensor lumen 10L (dashed line) for accommodating the sensor 70 is formed inside the sensor tube 10. The sensor lumen 10L is a lumen for the sensor 70.

In the longitudinal direction of the catheter 1, the distal end of the sensor tube 10 is located at the same position as the distal end of the RX tube 30 or slightly closer to the proximal end side than the distal end of the RX tube 30. A distal end opening 101 communicating between the distal end of the sensor lumen 10L and the outside is formed at the distal end of the sensor tube 10. The distal end opening 101 is a fluid discharge port for setting the inside of the sensor lumen 10L to a wet state. The proximal end of the sensor tube 10 is located closer to the proximal end side than the proximal end of the OTW lumen 20L and the proximal end of the RX tube 30 in the longitudinal direction of the catheter 1. The first reinforcing member 61, the branching connector 60, the cylindrical member 64, and the connector 65 are attached on the proximal end side of the sensor tube 10 from the distal end side toward the proximal end side. Details will be described below. A fluid supply portion 66 is attached to the connector 65, and a proximal end opening 102 communicating between the proximal end of the sensor lumen 10L and the outside is formed in the fluid supply portion 66. The proximal end opening 102 is a fluid supply port to the sensor lumen 10L.

As illustrated in FIG. 1, the sensor tube 10 includes a distal end side tube 11 provided on the distal end side and a proximal end side tube 12 provided closer to the proximal end side than the distal end side tube 11. Both the distal end side tube 11 and the proximal end side tube 12 are hollow cylindrical members (tubular bodies) having an elongated outer shape. The distal end side tube 11 and the proximal end side tube 12 are connected to each other inside the first outer tube 50 in the longitudinal direction.

Specifically, each of the distal end side tube 11 and the proximal end side tube 12 forms a part of the sensor lumen 10L.

The OTW tube 20 is a hollow cylindrical member (tubular body) having an elongated outer shape. On the distal end side of the branching connector 60, the OTW tube 20 extends linearly along the longitudinal direction of the catheter 1 in parallel with the sensor tube 10 and the RX tube 30. Inside the OTW tube 20, an OTW lumen 20L (dashed line) for accommodating a therapeutic device (e.g., a plasma guide wire or a penetration guide wire) is formed. The OTW lumen 20L has no proximal end opening in a portion located in the living body lumen when the catheter 1 is used and is what is called an over-the-wire (OTW) type lumen.

The distal end of the OTW tube 20 is located closer to the proximal end side than the distal end of the sensor tube 10 and the distal end of the RX tube 30 in the longitudinal direction of the catheter 1. A distal end opening 201 communicating between the distal end of the OTW lumen 20L and the outside is formed at the distal end of the OTW tube 20. The distal end opening 201 is a device projection port allowing a therapeutic device to project toward a living body tissue. Since the distal end portion of the OTW tube 20 is obliquely cut, the distal end opening 201 is oriented in a direction intersecting with the longitudinal direction of the catheter 1. Thus, when the catheter 1 is used, the therapeutic device can easily reach a living body tissue present around the catheter 1. The proximal end of the OTW tube 20 is located closer to the distal end side than the proximal end of the sensor tube 10 and closer to the proximal end side than the proximal end of the RX tube 30 in the longitudinal direction of the catheter 1. The first reinforcing member 61, the branching connector 60, the second reinforcing member 62, the third reinforcing member 63, and the connector 25 are attached on the proximal end side of the OTW tube 20 from the distal end side toward the proximal end side. Details will be described later. A proximal end opening 202 communicating between the proximal end of the OTW lumen 20L and the outside is formed at the connector 25. The proximal end opening 202 is a device insertion port allowing a therapeutic device to be inserted into the OTW lumen 20L.

As illustrated in FIG. 1, the OTW tube 20 includes a distal end side tube 21 provided on the distal end side and a proximal end side tube 22 provided closer to the proximal end side than the distal end side tube 21. Both the distal end side tube 21 and the proximal end side tube 22 are hollow cylindrical members (tubular bodies) having an elongated outer shape. The distal end side tube 21 and the proximal end side tube 22 are connected to each other inside the first outer tube 50 in the longitudinal direction. Specifically, each of the distal end side tube 21 and the proximal end side tube 22 forms a part of the OTW lumen 20L.

The RX tube 30 is a hollow cylindrical member (tubular body) having an elongated outer shape. The RX tube 30 extends linearly along the longitudinal direction of the catheter 1 in parallel with the sensor tube 10 and the OTW tube 20. An RX lumen 30L (dashed line) for accommodating a work hose wire is formed inside the RX tube 30.

The distal end of the RX tube 30 is located at the same position as the distal end of the sensor tube 10 or slightly closer to the distal end side than the distal end of the sensor tube 10 in the longitudinal direction of the catheter 1. The hollow distal tip 40 is joined to the distal end portion of the RX tube 30. A distal end opening 301 communicating between the distal end of the RX lumen 30L and the outside is formed at the distal end of the distal tip 40. The distal end opening 301 is a wire insertion port allowing a work hose wire to be inserted into the RX lumen 30L. The proximal end of the RX tube 30 is located closer to the distal end side than the proximal end of the sensor tube 10 and the proximal end of the OTW tube 20 in the longitudinal direction of the catheter 1. A proximal end opening 302 communicating between the proximal end of the RX lumen 30L and the outside is formed at the proximal end of the RX tube 30. The proximal end opening 302 is a wire drawing port for drawing out the work hose wire to the outside. Since the proximal end of the RX tube 30 is obliquely cut, the proximal end opening 302 is oriented in a direction intersecting with the longitudinal direction of the catheter 1. Thus, when the catheter 1 is used, the work hose wire can be easily drawn out from the proximal end opening 302.

The distal tip 40 has radiopacity and is a cylindrical member in which the outer diameter expands from the distal end side toward the proximal end side. The distal tip 40 is joined to the distal end portion of the RX tube 30 and is thus located at the distal end of the catheter 1 and proceeds in the living body lumen prior to the other members. The inner cavity of the distal tip 40 communicates with the RX lumen 30L of the RX tube 30, and the distal end opening 301 communicating between the distal end of the RX lumen 30L and the outside is formed at the distal end of the distal tip 40 as described above.

The first marker 41 and the second marker 42 are annular members having radiopacity. The first marker 41 is provided such that the proximal end of the first marker 41 and the proximal end of the distal tip 40 are located at the same position in the longitudinal direction of the catheter 1. The first marker 41 is embedded between the outer peripheral surface of the RX tube 30 and the inner peripheral surface of the distal tip 40. The second marker 42 is provided such that the proximal end of the second marker 42 and the distal end of the distal end opening 201 are located at the same position in the longitudinal direction of the catheter 1. The second marker 42 is joined to the outer peripheral surface of the RX tube 30. For joining the first marker 41 and the second marker 42, for example, joining between resins by thermal melting or joining with an adhesive such as an epoxy-based adhesive can be adopted. The second marker 42 is visible to the naked eye. As described above, by providing the first marker 41 and the second marker 42 on the RX tube 30, it is possible to prevent the first marker 41 and the second marker 42 from interfering with sensing (acquisition of image information) by the sensor 70.

As illustrated in FIG. 3A, in the transverse section taken along the line A-A, the sensor tube 10 (i.e., the distal end side tube 11) and the RX tube 30 are provided, and the outer peripheral surfaces thereof are joined to each other. As illustrated in FIG. 3B, in the transverse section taken along the line B-B, the sensor tube 10 (i.e., the distal end side tube 11), the OTW tube 20 (i.e., the distal end side tube 21), and the RX tube 30 are provided, and the outer peripheral surfaces thereof are joined to each other. As illustrated in FIG. 3C, in the transverse section taken along the line C-C, the sensor tube 10 (i.e., the distal end side tube 11), the OTW tube 20 (i.e., the distal end side tube 21), and the RX tube 30 are covered with the first outer tube 50. Specifically, the outer peripheral surfaces of the three tubes 10, 20, and 30 are covered with the first outer tube 50 formed by melting, and thus the three tubes 10, 20, and 30 are integrally fixed. As illustrated in FIG. 3D, in the transverse section taken along the line D-D, the sensor tube 10 (i.e., the distal end side tube 11), the OTW tube 20 (i.e., the proximal end side tube 22), and the RX tube 30 are covered with the first outer tube 50, as in FIG. 3C. As illustrated in FIG. 3E, in the transverse section taken along the line E-E, the sensor tube 10 (i.e., the proximal end side tube 12) and the OTW tube 20 (i.e., the proximal end side tube 22) are covered with the second outer tube 80. Specifically, the outer peripheral surfaces of the two tubes 10 and 20 are covered with the second outer tube 80 formed by melting, and thus the two tubes 10 and 20 are integrally fixed.

In the A-A transverse section and the B-B transverse section, the sensor tube 10, the OTW tube 20, and the RX tube 30 may be joined to each other by using any bonding material such as an epoxy-based adhesive or may be welded by heat. In the A-A transverse section, the B-B transverse section, the C-C transverse section, and the D-D transverse section, a height LY of the catheter 1 is greater than a width LZ of the catheter 1. On the other hand, in the E-E transverse section, the height LY of the catheter 1 is smaller than the width LZ of the catheter 1. As illustrated in FIGS. 3A to 3E, the size relationship among the outer diameters of the three tubes 10, 20, and 30 is the outer diameter of the sensor tube 10>the outer diameter of the OTW tube 20>the outer diameter of the RX tube 30. In addition, the size relationship among the inner diameters (lumens) of the three tubes 10, 20, and 30 is the inner diameter of the sensor lumen 10L>the inner diameter of the OTW lumen 20L >the inner diameter of the RX lumen 30L. However, the size relationship among the outer diameters and the inner diameters is merely an example and may be arbitrarily changed.

The A-A transverse section and the B-B transverse section, i.e., the outer shape of the catheter 1 closer to the distal end side than the first outer tube 50, is the shape along the contour of the two tubes 10 and 30 (or the three tubes 10, 20, and 30) arranged adjacent to each other, and a constriction (recess portion) is formed in the adjacent portion of each tube. The C-C transverse section and the D-D transverse section, i.e., the outer shape of the catheter 1 in the portion covered with the first outer tube 50, is a triangular shape with round corners (a round-corner triangular shape). The E-E transverse section, i.e., the outer shape of the catheter 1 in the portion covered with the second outer tube 80, is elliptical.

Returning to FIG. 1, the description will be continued. The three tubes 10, 20, and 30 (i.e., the sensor tube 10, the OTW tube 20, and the RX tube 30) are fixed by the three tubes 90, 50, and 80.

The heat-shrinkable tube 90 is provided between the first outer tube 50 and the second outer tube 80 in the longitudinal direction of the catheter 1. The heat-shrinkable tube 90 covers the sensor tube 10 (i.e., a part of the proximal end side tube 12 on the distal end side) and the OTW tube 20 (i.e., a part of the proximal end side tube 22 on the distal end side) and bundles the two tubes 10 and 20. The heat-shrinkable tube 90 does not cover the RX tube 30, and the RX tube 30 is provided along the outer peripheral surface of the heat-shrinkable tube 90 in a state where the outer peripheral surface of the heat-shrinkable tube 90 is in contact with the outer peripheral surface of the RX tube 30. The distal end of the heat-shrinkable tube 90 is located closer to the proximal end side than the distal end of the first outer tube 50 and closer to the distal end side than the proximal end opening 302. Specifically, the distal end portion of the heat-shrinkable tube 90 is covered with the first outer tube 50. The proximal end of the heat-shrinkable tube 90 is located closer to the proximal end side than the distal end of the second outer tube 80 and closer to the distal end side than the first reinforcing member 61. That is, the proximal end portion of the heat-shrinkable tube 90 is covered with the second outer tube 80. In other words, an intermediate portion of the heat-shrinkable tube 90 in the longitudinal direction of the catheter 1 is not covered with the first outer tube 50 or the second outer tube 80.

The first outer tube 50 is located closer to the distal end side than the heat-shrinkable tube 90 in the longitudinal direction of the catheter 1. The first outer tube 50 is provided in a section which is closer to the proximal end side than the distal end opening 201 and in which the three tubes 10, 20, and 30 extend side by side. In the example of FIG. 1, the distal end of the first outer tube 50 is located near the center between the distal end opening 201 and the proximal end opening 302. The proximal end of the first outer tube 50 is located near the proximal end of the proximal end opening 302. As described above, the first outer tube 50 may be provided at a position away from the distal end opening 201 toward the proximal end side. Thus, the first outer tube 50 may be prevented from interfering with sensing (acquisition of image information) by the sensor 70 inserted into the sensor lumen 10L. The first outer tube 50 covers and fixes the distal end portion of the heat-shrinkable tube 90, the sensor tube 10 (i.e., a part of the distal end side tube 11 on the proximal end side) exposed from the distal end of the heat-shrinkable tube 90, the OTW tube 20 (i.e., a part of the distal end side tube 21 on the proximal end side and a part of the proximal end side tube 22 on the distal end side) exposed from the distal end of the heat-shrinkable tube 90, and the RX tube 30. As illustrated in FIGS. 3C and 3D, the first outer tube 50 has a triangular outer shape with round corners and has a thick portion formed by melting along the outer peripheral surfaces of the three tubes 10, 20, and 30.

The second outer tube 80 is provided closer to the proximal end side than the heat-shrinkable tube 90 in the longitudinal direction of the catheter 1. The second outer tube 80 is provided in a section which is closer to the proximal end side than the proximal end opening 302 and in which the two tubes 10 and 20 extend side by side. In the example of FIG. 1, the distal end of the second outer tube 80 is located at a portion slightly away from the proximal end opening 302 toward the proximal end side. The proximal end of the second outer tube 80 is located inside the first reinforcing member 61. The second outer tube 80 covers and fixes the proximal end portion of the heat-shrinkable tube 90, the sensor tube 10 (i.e., a part of the proximal end side tube 12 on the proximal end side) exposed from the proximal end of the heat-shrinkable tube 90, and the OTW tube 20 (i.e., a part of the proximal end side tube 22) exposed from the proximal end of the heat-shrinkable tube 90. As illustrated in FIG. 3E, the second outer tube 80 has an elliptical outer shape and has a thick portion formed by melting along the outer peripheral surfaces of the two tubes 10 and 20.

The branching connector 60 is a member having a bifurcated inner cavity and is provided on the proximal end side of the catheter 1. The OTW tube 20 is inserted into one inner cavity of the branching connector 60. The sensor tube 10 is inserted into the other inner cavity of the branching connector 60. The first reinforcing member 61 is a hollow cylindrical member provided closer to the distal end side than the branching connector 60. The first reinforcing member 61 covers the outer periphery of the second outer tube 80, which bundles the sensor tube 10 and the OTW tube 20, and thus reinforces the distal end side of the branching connector 60.

The second reinforcing member 62 is a hollow cylindrical member provided closer to the proximal end side than one branch of the branching connector 60. The second reinforcing member 62 covers the outer periphery of the OTW tube 20 inserted into the branching connector 60 and thus reinforces the proximal end side of the branching connector 60. The third reinforcing member 63 is a hollow cylindrical member provided closer to the distal end side than the connector 25. The third reinforcing member 63 covers the outer periphery of the OTW tube 20 inserted into the connector 25 and thus reinforces the distal end side of the connector 25. The connector 25 is a member joined to the proximal end portion of the OTW tube 20. The connector 25 includes a pair of blade portions to be gripped by the professional. The proximal end opening 202 (device insertion port) communicating between the proximal end of the OTW lumen 20L and the outside is formed at the proximal end of the connector 25.

The cylindrical member 64 is a hollow cylindrical member provided closer to the proximal end side than the other branch of the branching connector 60. The cylindrical member 64 covers the outer periphery of the sensor tube 10 inserted into the branching connector 60 and thus reinforces the proximal end side of the branching connector 60. The connector 65 is a member joined to the proximal end portion of the sensor tube 10. A housing for accommodating a connection terminal 75 of the sensor 70 is provided on the proximal end side of the connector 65. The fluid supply portion 66, in which the proximal end opening 102 communicating between the proximal end of the sensor lumen 10L and the outside is formed, is provided on the outer peripheral surface of the connector 65.

The sensor 70 (FIG. 2) is an imaging sensor that acquires image information. As illustrated in FIG. 2, the sensor 70 includes a main body part 71, a probe 72, and the connection terminal 75. The main body part 71 is an elongated member extending along the longitudinal direction of the catheter 1. A driving cable (coaxial line) electrically connecting the probe 72 and the connection terminal 75 is built inside the main body part 71. The probe 72 includes an ultrasonic probe (also referred to as an ultrasonic vibrator, a piezoelectric body, an ultrasonic transmission/reception element, or an ultrasonic element) that transmits ultrasonic waves toward a living body tissue and receives ultrasonic waves propagated through the living body tissue and reflected. The probe 72 is also referred to as an imaging core or a transducer. The connection terminal 75 is a terminal that electrically connects the sensor 70 to a console terminal provided outside. The connection terminal 75 is provided at the proximal end of the main body part 71 and is accommodated in the housing of the connector 65.

The sensor 70 is electrically connected to an external console terminal via the connection terminal 75, receives power supplied from the console terminal, and outputs a detection signal by the probe 72 to the console terminal. Thus, the console terminal can display image information based on the detection signal of the probe 72. As illustrated in FIG. 2, the sensor 70 is fixed to the connector 65. Furthermore, as indicated by the white arrow in FIG. 2, the professional grips the connector 65 and slides the connector 65 in the front-rear direction (the direction of the white arrow) and thus can move the position of the probe 72 of the sensor 70 within a range MR from the distal end of the sensor lumen 10L to the distal end of the first outer tube 50, in other words, within the prescribed range MR including the distal end opening 201. Hereinafter, the range MR is also referred to as the “movable range MR”. Further, a portion of the catheter 1 which is particularly suitable for sensing (acquisition of image information) by the sensor 70 is also referred to as an “acoustic window AW”. As illustrated in FIG. 2, the acoustic window AW is a section between the first marker 41 and the second marker 42 in the catheter 1.

The distal end side tube 11 of the sensor tube 10, the distal end side tube 21 of the OTW tube 20, and the RX tube 30 can be formed of a flexible material, e.g., a thermoplastic resin such as a polyethylene resin, a polypropylene resin, or polyurethane, polyvinyl chloride, ethylene-vinyl acetate copolymers, cross-linked ethylene-vinyl acetate copolymers, polyamide elastomer, polyolefin elastomer, polyurethane elastomer, silicone rubber, or latex rubber. The distal end side tube 11 of the sensor tube 10, the distal end side tube 21 of the OTW tube 20, and the RX tube 30 may be formed of the same material or different materials.

The proximal end side tube 12 of the sensor tube 10 and the proximal end side tube 22 of the OTW tube 20 can be formed of, for example, a resin having a high rigidity such as a nylon resin, a polyester resin, or a PEEK resin. The melting points of the proximal end side tube 12 of the sensor tube 10 and the proximal end side tube 22 of the OTW tube 20 are higher than the melting points of the above-described tubes 11, 21, and 30. The proximal end side tube 12 of the sensor tube 10 and the proximal end side tube 22 of the OTW tube 20 may be formed of the same material or different materials.

In the catheter 1 according to the present embodiment, gradual changes in the rigidity of the catheter 1 is achieved by providing the section in which a part of the RX tube 30 having flexibility on the proximal end side is overlapped with the proximal end side tube 12 and the proximal end side tube 22 having a high rigidity (FIG. 1). Gradual changes in the rigidity of the catheter 1 may be rephrased as reduction in a rigidity gap of the catheter 1. As a result, kink of the catheter 1 can be suppressed. Further, any one or more of the distal end side tube 11 and the proximal end side tube 12 of the sensor tube 10, the distal end side tube 21 and the proximal end side tube 22 of the OTW tube 20, and the RX tube 30 may have a multilayer structure in which the tubes having different materials are stacked.

The distal tip 40, the first marker 41, and the second marker 42 can be formed of a resin material or a metal material having radiopacity. For example, when a resin material having radiopacity is used, it can be formed by mixing a radiopaque material such as bismuth trioxide, tungsten, or barium sulfate with a polyamide resin, a polyolefin resin, a polyester resin, a polyurethane resin, a silicone resin, or a fluororesin. For example, when a metal material having radiopacity is used, it can be formed of gold, platinum, tungsten, or an alloy containing these elements (e.g., a platinum-nickel alloy). The distal tip 40, the first marker 41, and the second marker 42 may be formed of the same material or different materials.

The branching connector 60, the first reinforcing member 61 to the third reinforcing member 63, the cylindrical member 64, the connector 65, and the connector 25 can be formed of a known resin material. The branching connector 60, the first reinforcing member 61 to the third reinforcing member 63, the cylindrical member 64, the connector 65, and the connector 25 may be formed of the same material or different materials.

The heat-shrinkable tube 90 is formed of a nylon-based elastomer resin (e.g., polyamide elastomer) having thermoplasticity. The heat-shrinkable tube 90 has a property of shrinking without melting when heated in a prescribed temperature range. Further, the heat-shrinkable tube 90 improves adhesiveness (the property of easily sticking to another substance) during heating as compared to that during non-heating. The heat-shrinkable tube 90 may be formed of polyolefin, FEP (Fluorinated Ethylene Propylene), or silicone.

The first outer tube 50 and the second outer tube 80 are formed of a nylon-based elastomer resin having thermoplasticity. Unlike the heat-shrinkable tube 90, the first outer tube 50 and the second outer tube 80 have the property of melting during heating. In the example of the present embodiment, a resin having lower shore hardness than the second outer tube 80 is used for the first outer tube 50. However, the first outer tube 50 and the second outer tube 80 may be formed of the same material or different materials.

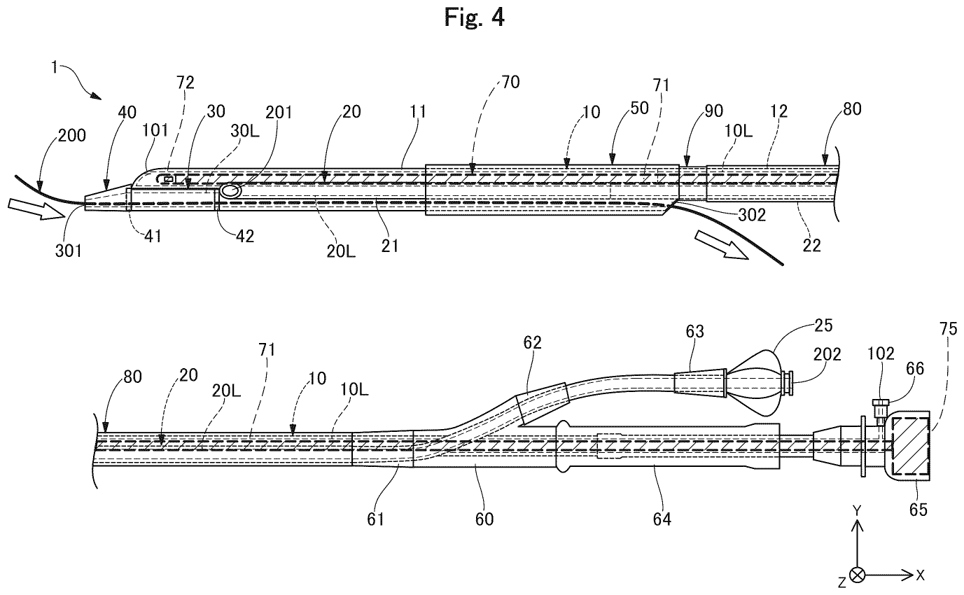

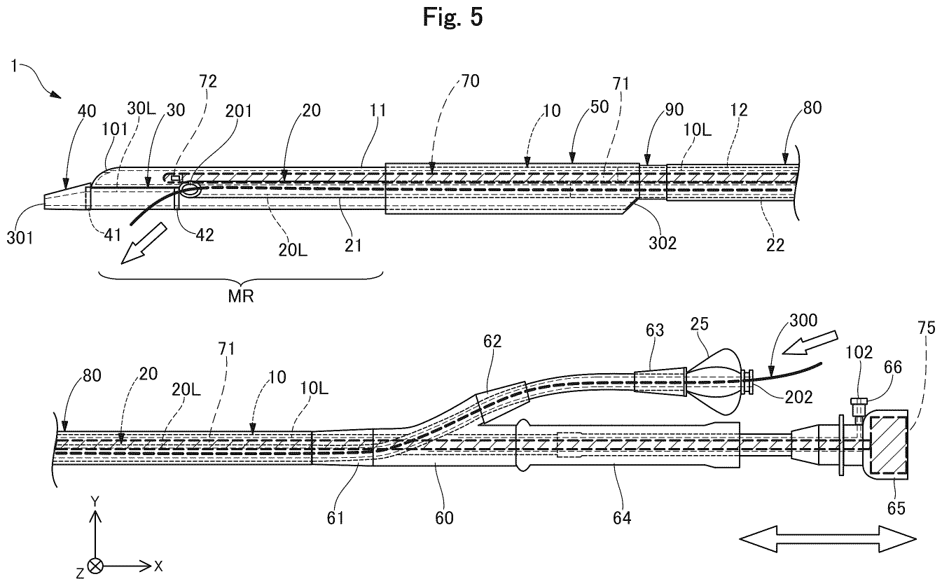

FIGS. 4 and 5 are diagrams illustrating a method of using the catheter 1. In the following steps a1 to a6, the case of recanalization of a CTO (lesion) generated in a blood vessel by the antegrade approach will be described as an example. The catheter 1 may be used in a retrograde approach and may be used for operation other than recanalization of a CTO.

(a1) The professional inserts the work hose wire 200 into a blood vessel and delivers the distal end portion of the work hose wire 200 to the vicinity of the CTO. (a2) The professional inserts the proximal end portion of the work hose wire 200 from the distal end opening 301 of the catheter 1, passes it through the RX lumen 30L, and draws it out from the proximal end opening 302 of the catheter 1 (FIG. 4). (a3) The professional pushes the catheter 1 into the blood vessel along the work hose wire 200 and delivers the distal end portion of the catheter 1 to the vicinity of the CTO. In the step a3, the catheter 1 may be delivered to the vicinity of the CTO by passing the catheter 1 through a guiding catheter that is previously inserted into the blood vessel along the work hose wire 200. (a4) While gripping the connector 65, sliding the connector 65 in the front-rear direction (FIG. 5: the direction of the white arrow), and thus adjusting the position of the probe 72 of the sensor 70 within the movable range MR, the professional checks the image displayed on the console terminal and thus matches the positions and orientations of the CTO and the distal end opening 201. The position refers to the position in the extending direction of the blood vessel, and the orientation refers to the orientation in the circumferential direction of the inner wall of the blood vessel. (a5) The professional inserts the distal end portion of a therapeutic device 300 from the proximal end opening 202 of the catheter 1, inserts the distal end portion into the OTW lumen 20L, and causes the distal end portion to protrude from the distal end opening 201 of the catheter 1 (FIG. 5). (a6) The professional treats the CTO using the therapeutic device 300 while adjusting the position of the probe 72 of the sensor 70 within the movable range MR as necessary and checking the image displayed on the console terminal. As described above, any device such as a plasma guide wire or a penetration guide wire can be used as the therapeutic device 300.

The sensor tube 10, the OTW tube 20, and the RX tube 30 are also collectively referred to as a “shaft”. The sensor tube 10 (the distal end side tube 11 and the proximal end side tube 12) corresponds to a “first tube”, and the sensor lumen 10L corresponds to a “first lumen”. The RX tube 30 corresponds to a “second tube”, and the RX lumen 30L corresponds to a “second lumen”. According to the present embodiment, “the same” and “equal” are not limited to the case of an exact match and have a meaning of allowing differences due to a manufacturing error, or the like. Furthermore, “constant” is synonymous with “substantially constant” and means substantially constant while allowing a deviation due to a manufacturing error, or the like.

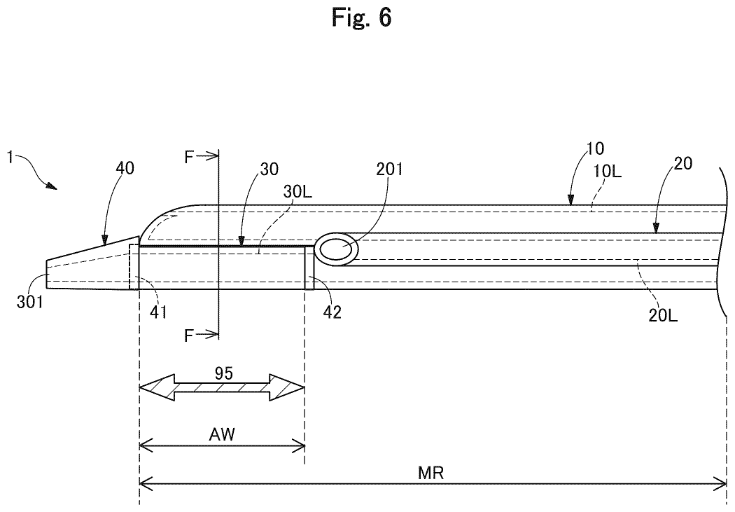

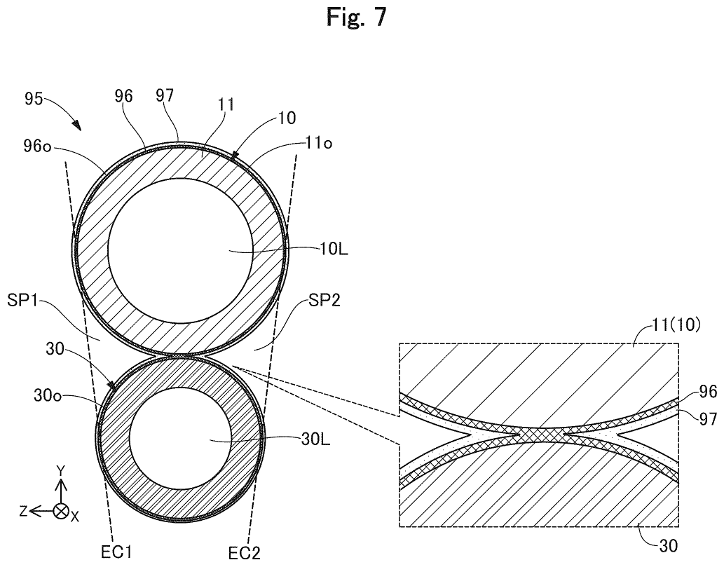

FIG. 6 is an enlarged view of a part of the catheter 1 on the distal end side. FIG. 7 is a transverse sectional view of the catheter 1 taken along the line F-F in FIG. 6. FIG. 7 (the F-F transverse section) is a transverse section of the catheter 1 including the acoustic window AW. A part of the transverse sectional view is illustrated in an enlarged manner within a balloon indicated by a dashed line of FIG. 7. The coupling between the sensor tube 10 and the OTW tube 20 on the distal end side of the catheter 1 will be further described with reference to FIGS. 6 and 7.

As illustrated in FIG. 6, in the longitudinal direction of the catheter 1, a section between the first marker 41 and the second marker 42 of the RX tube 30 (the second tube) is the acoustic window AW particularly suitable for sensing (acquisition of image information) by the sensor 70. As illustrated in FIGS. 6 and 7, in the acoustic window AW, a wall portion of the RX tube 30 is present over the entire circumferential direction. In other words, in the section where the acoustic window AW is present, the RX tube 30 is not provided with a hole communicating between the inside and the outside of the RX lumen 30L, a notch, or the like.

In the catheter 1 according to the present embodiment, a coupling portion 95 is provided in the entirety between the first marker 41 and the second marker 42 (in other words, the entire acoustic window AW in the longitudinal direction). The coupling portion 95 is a portion where the distal end side tube 11 (the first tube) of the sensor tube 10 and the RX tube 30 (the second tube) are coupled to each other with a coating agent. Hereinafter, the distal end side tube 11 of the sensor tube 10 and the RX tube 30 are also simply referred to as the “tubes 10 and 30” or the “first and second tubes 10 and 30”.

According to the present embodiment, the coating agent includes a base agent and a top agent. The base agent is an agent for improving the fixability of the top agent to the resin tube (i.e., the distal end side tube 11 and the RX tube 30). As the base agent, for example, an acrylic resin, acrylamide, carboxylic acid, or the like can be used. The top agent is an agent for imparting hydrophilicity. As the top agent, for example, a well-known hydrophilic resin such as hyaluronic acid can be used. Hereinafter, the layer formed by applying the coating agent is also referred to as a “coating layer”. As used herein, a “coating agent” refers to a liquid substance that, when applied to the surfaces of the first and second tubes and subsequently dried or cured, forms a thin film that adheres to and mechanically couples the tubes. The coating agent is distinct from traditional adhesives in that the coating agent can be applied as a thin layer without filling the entire space between the tubes and cured without significantly stiffening the coupled portion.

The coating layer according to the present embodiment includes an undercoat layer 96 formed by applying the base agent and an overcoat layer 97 formed by applying the top agent. The undercoat layer 96 is in contact with an outer peripheral surface 11o of the distal end side tube 11 and an outer peripheral surface 30o of the RX tube 30 along the outer peripheral surfaces 11o and 30o and thinly covers the outer peripheral surfaces 11o and 30o (FIG. 7). As illustrated within the balloon indicated by a dashed line of FIG. 7, the undercoat layer 96 is interposed between the outer peripheral surface 11o of the distal end side tube 11 and the outer peripheral surface 30o of the RX tube 30 at the portion where the distal end side tube 11 and the RX tube 30 are adjacent to each other.

The overcoat layer 97 is in contact with an outer peripheral surface 96o of the undercoat layer 96 along the outer peripheral surface 96o and thinly covers the outer peripheral surface 96o (FIG. 7). As illustrated within the balloon indicated by a dashed line of FIG. 7, the overcoat layer 97 is not interposed between the outer peripheral surface 11o of the distal end side tube 11 and the outer peripheral surface 30o of the RX tube 30. That is, in the example of FIG. 7, it can be said that the hydrophilic coating base agent couples the distal end side tube 11 of the sensor tube 10 and the RX tube 30.

In FIG. 7, common external tangent lines EC1 and EC2 of the distal end side tube 11 (the first tube) of the sensor tube 10 and the RX tube 30 (the second tube) are indicated by dashed lines. The common external tangent line EC1 is also referred to as the “first common external tangent line EC1”, and the common external tangent line EC2 is also referred to as the “second common external tangent line EC2”. As illustrated in the drawing, a space SP1 is formed between the outer peripheries of the distal end side tube 11 and the RX tube 30 and the first common external tangent line EC1. Similarly, a space SP2 is formed between the outer peripheries of the distal end side tube 11 and the RX tube 30 and the second common external tangent line EC2. The presence of the spaces SP1 and SP2 means that, in the transverse section of the catheter 1 including the acoustic window AW, the contour of the catheter 1 has a constriction (recess portion) at the adjacent portion where the two tubes 11 and 30 are arranged adjacent to each other.

As a method for forming the coupling portion 95, in other words, a method for coupling the tubes 10 and 30 with the coating agent, for example, a method described in b1 to b8 below can be adopted. (b1) A liquid of the base agent and a liquid of the top agent are prepared. (b2) In a state where the distal end side tube 11 (the first tube) of the sensor tube 10 and the RX tube 30 (the second tube) are arranged side by side, at least the entire portion where the coupling portion 95 is to be formed is immersed in the liquid of the base agent. In the step b2, the entirety of the catheter 1 closer to the distal end side than the second marker 42 may be immersed in the base agent, or the entirety of the catheter 1 from the distal end to the proximal end may be immersed in the base agent. At this time, the distal tip 40, the first marker 41, and the second marker 42 may have been already formed at the distal end portion of the RX tube 30, or the tubes 10, 20, and 30 may be fixed by the first outer tube 50 or the like. (b3) Due to surface tension, the liquid of the base agent is interposed in the gap (FIG. 7: between the outer peripheral surface 11o and the outer peripheral surface 30o) of the portion where the tube 10 and the tube 30 are adjacent to each other. (b4) The thickness of the undercoat layer 96 is controlled by sucking up the excess liquid of the base agent on the outer peripheral surfaces of the tube 10 and the tube 30 using a cleaning tissue or the like. (b5) The undercoat layer 96 is formed by drying the liquid of the base agent. (b6) As in the step b2, at least the entire portion where the coupling portion 95 is to be formed is immersed in the liquid of the top agent. (b7) The thickness of the overcoat layer 97 is controlled by sucking up the excess liquid of the top agent on the outer peripheral surface of the undercoat layer 96 using a cleaning tissue or the like. (b8) The overcoat layer 97 is formed by drying the liquid of the top agent.

The undercoat layer 96 formed of the base agent is thin due to the property of the base agent for “improving the fixability of the top agent to the tubes 10 and 30”, but has the properties of high adhesiveness to the tubes 10 and 30, strength against a mechanical load, and does not impair the flexibility of the tubes 10 and 30 even after the undercoat layer 96 (film) is formed. According to the present embodiment, since the distal end side tube 11 of the sensor tube 10 and the RX tube 30 are coupled to each other with the undercoat layer 96 formed of the base agent, it is possible to avoid disadvantages caused when coupling is provided using an adhesive, for example, a decrease in the flexibility and the operability due to curing of the coupling portion 95 and a decrease in the acoustic characteristics in the coupling portion 95 (i.e., the acoustic window AW).

Specifically, in the case of coupling using an adhesive, the flexibility and the acoustic characteristics of the catheter at the location where the adhesive is applied are reduced due to the curing of the adhesive itself. In addition, coating layers (such as the undercoat layer 96 and the overcoat layer 97) for imparting hydrophilicity and hydrophobicity are generally provided in the catheter in order to improve torquability and blood vessel followability; however, in the case of coupling using an adhesive, a coating layer is further formed on the adhesive layer, and thus the flexibility and the acoustic characteristics of the catheter are further deteriorated. Further, in the case of coupling using an adhesive, it is difficult to control the thickness of the adhesive layer by sucking the adhesive using a cleaning tissue due to the property of the adhesive, and therefore a thick adhesive layer is formed, and the above-described spaces SP1 and SP2 cannot be provided. Thus, the thick adhesive layer further reduces the flexibility and the acoustic characteristics of the catheter and increases the outer diameter of the catheter in the corresponding portion. Such deterioration of the acoustic characteristics causes problems such as darkening of an image obtained by the sensor and occurrence of a defect.

In this regard, in the catheter 1 according to the present embodiment, since the distal end side tube 11 of the sensor tube 10 and the RX tube 30 are coupled to each other with the undercoat layer 96 formed of the base agent without using the adhesive having the above-described disadvantage, a decrease in the flexibility and the operability due to curing of the coupling portion 95 and a decrease in the acoustic characteristics in the coupling portion 95 (i.e., the acoustic window AW) may be avoided. Furthermore, as is clear from the steps b1 to b8, according to the present embodiment, since heat is not applied when the coupling portion 95 (the undercoat layer 96 and the overcoat layer 97) is formed, deformation of the tubes 10 and 30 due to heat may be suppressed. Since the section closer to the proximal end side than the coupling portion 95 and closer to the distal end side than the first outer tube 50 does not function as the acoustic window AW, the three tubes 10, 20, and 30 may be coupled by thermal welding in the section.

As described above, with the catheter 1 according to the first embodiment, the distal end side tube 11 (the first tube) of the sensor tube 10 and the RX tube 30 (the second tube) can be coupled to each other with the coating agent without using heat fusion or an adhesive. Thus, the first tube and the second tube can be coupled to each other with the coating agent at a temperature below a melting point of the first tube and the second tube. For this reason, deformation of the first and second tubes 10 and 30, which occurs when the first and second tubes 10 and 30 are couped to each other by heat fusion, may be suppressed. In addition, it is possible to avoid disadvantages caused when coupling is provided using an adhesive, for example, a decrease in the flexibility and the operability due to curing of the coupling portion 95 and a decrease in the acoustic characteristics in the coupling portion 95. In addition, since the outer diameter of the catheter 1 can be reduced at the distal end portion of the catheter 1 provided with the coupling portion 95, the passing performance to the lesion can be improved, and the sliding with the combined device in the guiding catheter is reduced.

In addition, with the catheter 1 according to the first embodiment, since the sensor lumen 10L (the first lumen) allows the sensor 70 that acquires image information to be inserted to the sensor lumen 10L, it is possible to configure the catheter 1 (medical device) as a device that can also use the sensor 70, which acquires image information.

Furthermore, with the catheter 1 according to the first embodiment, in at least a part of the acoustic window AW, in which sensing (acquisition of image information) by the sensor 70 is performed, coupling is provided with a coating agent instead of an adhesive, and therefore, it is possible to prevent the sensing by the sensor 70 from being inhibited by the adhesive. In the example of FIG. 6, since coupling is provided in the entire acoustic window AW with the coating agent instead of the adhesive, it is possible to further prevent the sensing by the sensor 70 from being inhibited by the adhesive.

Further, with the catheter 1 according to the first embodiment, in the transverse section of the catheter 1 including the portion coupled with the coating agent, the spaces SP1 and SP2 are formed between the common external tangent lines EC1 and EC2 of the distal end side tube 11 (the first tube) of the sensor tube 10 and the RX tube 30 (the second tube) and the outer peripheries of the first and second tubes 10 and 30. In this way, the sensing by the sensor 70 can be performed more favorably by the spaces SP1 and SP2 that do not hinder the sensing by the sensor 70.

Further, with the catheter 1 according to the first embodiment, since the coating agent is the hydrophilic coating base agent, the hydrophilic coating can play a role of coupling the first and second tubes 10 and 30. In addition, with the catheter 1 according to the first embodiment, since the coupling portion 95 is provided in the entirety (the entire acoustic window AW) between the first marker 41 and the second marker 42, the entirety between the first marker 41 and the second marker 42 can be coupled with the coating agent.

### Second Embodiment

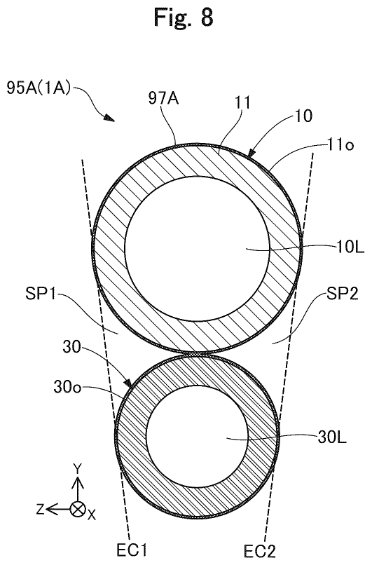

FIG. 8 is a transverse sectional view of a catheter 1A according to a second embodiment. FIG. 8 illustrates a transvers section of the catheter 1A including the acoustic window AW taken along the line F-F of FIG. 6. The catheter 1A according to the second embodiment includes a coupling portion 95A instead of the coupling portion 95 in the configuration described in the first embodiment.

The coupling portion 95A is a portion where the distal end side tube 11 (the first tube) of the sensor tube 10 and the RX tube 30 (the second tube) are coupled to each other with a coating agent including only one agent. The coating agent according to the second embodiment includes only one agent having both functions of a base agent and a top agent. As such a coating agent, for example, polyvinylpyrrolidone (PVP) or the like can be used. The coating layer according to the second embodiment includes a layer 97A formed by application of a coating agent. Similarly to the undercoat layer 96 according to the first embodiment, the layer 97A is in contact with the outer peripheral surfaces 11o and 30o of the tubes 10 and 30 along the outer peripheral surfaces 11o and 30o, thinly covers the outer peripheral surfaces 11o and 30o, and is interposed in the gap (FIG. 8: between the outer peripheral surface 11o and the outer peripheral surface 30o) at the portion where the tubes 10 and 30 are adjacent to each other. That is, according to the second embodiment, it can be said that the coating agent including only one agent couples the distal end side tube 11 of the sensor tube 10 and the RX tube 30.

As described above, the configuration of the coupling portion 95A can be variously changed, and the first and second tubes 10 and 30 may be coupled to each other using a coating agent including only one agent. The layer 97A has both functions of the base agent and the top agent and therefore, similarly to the undercoat layer 96 described in the first embodiment, although the layer 97A is thin, the layer 97A has the properties of high adhesiveness to the tubes 10 and 30, strength against a mechanical load, and does not impair the flexibility of the tubes 10 and 30 even after the layer 97A (film) is formed. Therefore, in the catheter 1A according to the second embodiment described above, too, the same effects as those of the first embodiment described above can be achieved.

### Third Embodiment

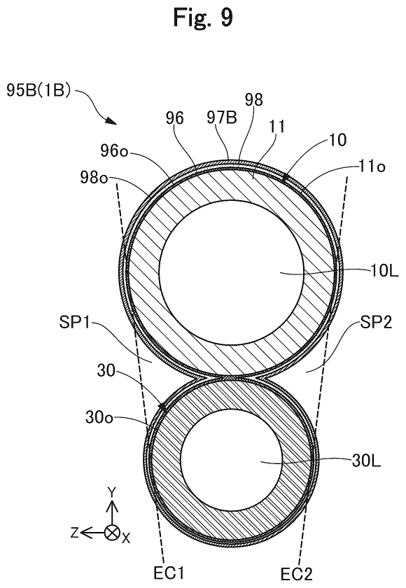

FIG. 9 is a transverse sectional view of a catheter 1B according to a third embodiment. FIG. 9 illustrates a transverse section of the catheter 1B including the acoustic window AW taken along the line F-F of FIG. 6. The catheter 1B according to the third embodiment includes a coupling portion 95B instead of the coupling portion 95 in the configuration described in the first embodiment.

The coupling portion 95B is a portion where the distal end side tube 11 (the first tube) of the sensor tube 10 and the RX tube 30 (the second tube) are coupled to each other with a coating agent including three agents. The coating agent according to the third embodiment further includes a second base agent provided between the base agent and the top agent in addition to the base agent and the top agent described in the first embodiment. As the second base agent, for example, an acrylic resin, acrylamide, carboxylic acid, or the like, can be used. Hereinafter, the base agent for the undercoat layer 96 is also referred to as a “first base agent”for distinction.

A coating layer according to the third embodiment includes the undercoat layer 96 formed by applying the first base agent, an intermediate layer 98 formed by applying the second base agent, and an overcoat layer 97B formed by applying the top agent. The undercoat layer 96 is the same as described in the first embodiment. The intermediate layer 98 is in contact with the outer peripheral surface 96o of the undercoat layer 96 along the outer peripheral surface 96o and thinly covers the outer peripheral surface 96o. The overcoat layer 97B is in contact with the outer peripheral surface 98o of the intermediate layer 98 along the outer peripheral surface 98o and thinly covers the outer peripheral surface 98o. That is, in the example of FIG. 9, too, it can be said that the hydrophilic coating base agent couples the distal end side tube 11 of the sensor tube 10 and the RX tube 30.

As described above, the configuration of the coupling portion 95B can be variously changed, and the first and second tubes 10 and 30 may be coupled to each other using the coating agent including three agents. Although the three agents are exemplified according to the present embodiment, a coating agent including four or more agents may be used. In the catheter 1B according to the third embodiment described above, too, the same effects as those of the first embodiment described above can be achieved. Further, in the catheter 1B according to the third embodiment, the first and second tubes 10 and 30 can be coupled to each other by using any coating agent having an agent composition corresponding to the materials of the distal end side tube 11 and the RX tube 30.

### Fourth Embodiment

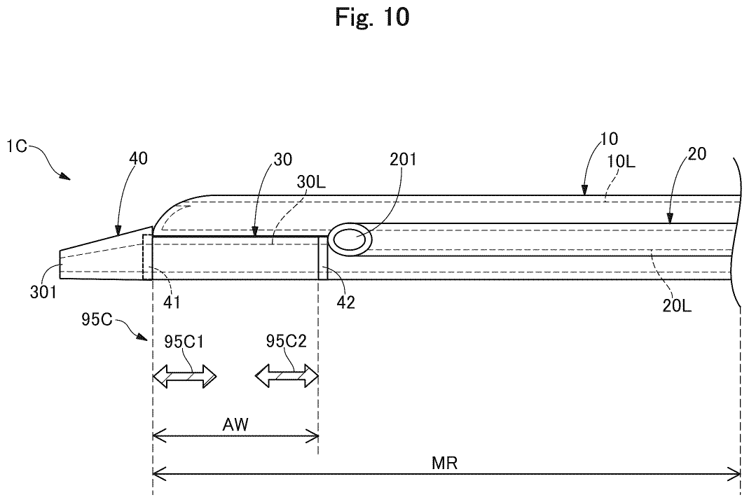

FIG. 10 is an enlarged view of a part of a catheter 1C on the distal end side according to a fourth embodiment. The catheter 1C according to the fourth embodiment includes a coupling portion 95C instead of the coupling portion 95 in the configuration described in the first embodiment. As described in the first embodiment, since the section between the first marker 41 and the second marker 42 corresponds to the acoustic window AW, the section between the first marker 41 and the second marker 42 is hereinafter also referred to as an “acoustic window section”.

The coupling portion 95C includes a first coupling portion 95C1 and a second coupling portion 95C2. The first coupling portion 95C1 is provided at the distal end portion of the acoustic window section. The second coupling portion 95C2 is provided at a position away from the first coupling portion 95C1 toward the proximal end side in the acoustic window section. In the illustrated example, the second coupling portion 95C2 is provided at the proximal end portion of the acoustic window section. The first coupling portion 95C1 and the second coupling portion 95C2 are portions where the distal end side tube 11 (the first tube) of the sensor tube 10 and the RX tube 30 (the second tube) are coupled to each other with the coating agent. Therefore, the tubes 10 and 30 are coupled to each other with the coating agent in the portion of the first coupling portion 95C1 and the portion of the second coupling portion 95C2. On the other hand, the tubes 10 and 30 are not coupled to each other in a portion located between the first coupling portion 95C1 and the second coupling portion 95C2 (in FIG. 10, the portion where the arrows hatched with diagonal lines are not illustrated). Hereinafter, this portion is also referred to as a “non-coupling portion”.

As described above, the configuration of the coupling portion 95C can be variously changed, and the first and second tubes 10 and 30 may be coupled to each other in at least a part of the acoustic window AW in the longitudinal direction of the catheter 1 and may include a non-coupling portion. Although the two coupling portions (the first coupling portion 95C1 and the second coupling portion 95C2) are illustrated in FIG. 10, the coupling portion 95C may include three or more coupling portions provided at positions away from each other. In the non-coupling portion, the RX tube 30 may be provided with a hole communicating between the inside and the outside of the RX lumen 30L or a notch. In the catheter 1C according to the fourth embodiment described above, too, the same effects as those of the first embodiment described above can be achieved. Further, with the catheter 1C according to the fourth embodiment, the coupling portions 95C1 and C2 of the first and second tubes 10 and 30 can be provided at two locations away from each other. Therefore, it is possible to further improve the flexibility and the acoustic characteristics of the catheter 1C in the non-coupling portion.

### Fifth Embodiment

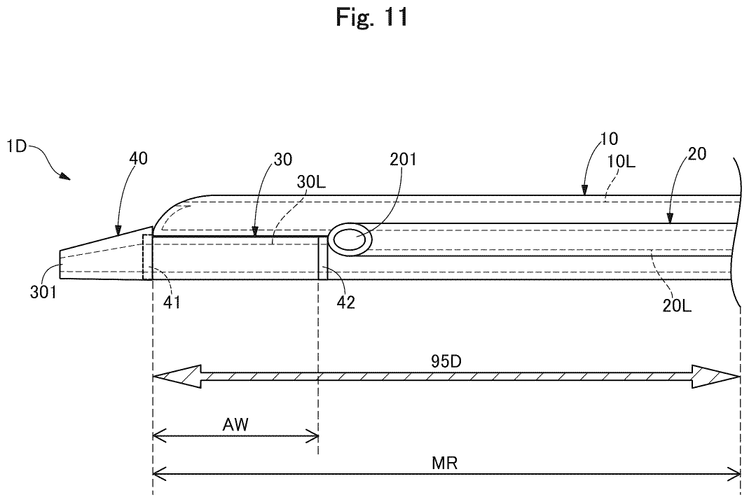

FIG. 11 is an enlarged view of a part of a catheter 1D on the distal end side according to a fifth embodiment. The catheter 1D according to the fifth embodiment includes a coupling portion 95D instead of the coupling portion 95 in the configuration described in the first embodiment. In addition to the section (acoustic window section) between the first marker 41 and the second marker 42, the coupling portion 95D further extends to a section closer to the proximal end side than the second marker 42 (closer to the proximal end side than the acoustic window AW). In the illustrated example, the coupling portion 95D is provided in the entire movable range MR of the sensor 70 from the distal end of the sensor lumen 10L to the distal end of the first outer tube 50.

As described above, the configuration of the coupling portion 95d can be

variously changed, and the coupling portion 95D may be provided in any range partially in the longitudinal direction of the catheter 1D. In the catheter 1D according to the fifth embodiment described above, too, the same effects as those of the first embodiment described above can be achieved.

### Sixth Embodiment

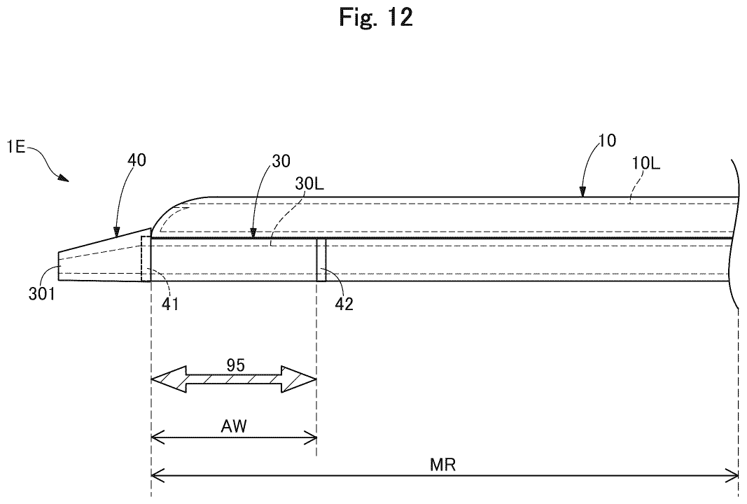

FIG. 12 is an enlarged view of a part of a catheter 1E on the distal end side according to a sixth embodiment. The catheter 1E according to the sixth embodiment does not include the OTW tube 20 and the OTW lumen 20L formed by the OTW tube 20 in the configuration described in the first embodiment.

As described above, the configuration of the catheter 1E can be variously changed and may be configured by omitting any part of the configuration described in the first embodiment. For example, the sensor tube 10 does not need to include the distal end side tube 11 and the proximal end side tube 12, and may be configured by one tube from the distal end to the proximal end. Further, the sensor tube 10 may be configured by combining three or more tubes. In the catheter 1E according to the sixth embodiment described above, too, the same effects as those of the first embodiment described above can be achieved.

### Modification of Present Embodiment

The disclosed embodiments are not limited to the above-described embodiments and can be implemented in various modes without departing from the gist of the disclosed embodiments, and for example, the following modifications are also possible.

### Modification 1

In the first to sixth embodiments, examples of the configurations of the catheters 1 and 1A to 1E have been described. However, the configurations of the catheters 1 and 1A to 1E can be variously changed.

For example, the coupling portion 95 may be provided only in a partial section between the first marker 41 and the second marker 42 (in other words, only in a partial section in the longitudinal direction of the acoustic window AW). For example, in the transverse section (F-F transverse section) of the catheter 1 including the acoustic window AW, the space SP1 does not need to be provided between the outer peripheries of the distal end side tube 11 and the RX tube 30 and the first common external tangent line EC1. In this case, for example, the undercoat layer 96 or the overcoat layer 97 may be formed in the portion corresponding to the space SP1 as described in FIG. 7, or the portion may be filled with a resin material or the like. Similarly, the space SP2 between the outer peripheries of the distal end side tube 11 and the RX tube 30 and the second common external tangent line EC2 may be omitted.

For example, the outer peripheral surfaces of the first outer tube 50, the heat-shrinkable tube 90, and the second outer tube 80, or the outer peripheral surface of the catheter 1 including these may be coated with a hydrophilic resin or a hydrophobic resin. This coating may be a coating layer (the undercoat layer 96, the overcoat layers 97 and 97B, the layer 97A, and the intermediate layer 98) formed of the above-described coating agent, or may be a coating layer different from the coating layer formed of the above-described coating agent. For example, the sensor 70 is built in the sensor lumen 10L of the sensor tube 10 and is not removable from the catheter 1. However, the sensor 70 may be configured to be removable from the catheter 1. That is, the catheter 1 does not need to include the sensor 70 as a constituent element.

For example, at least one of the distal tip 40, the first marker 41, and the second marker 42 may be omitted. For example, the shapes of the distal tip 40, the first marker 41, and the second marker 42 can be arbitrarily changed. The distal tip 40 may have a constant outer diameter from the distal end toward the proximal end, and the shape of the transverse section may be a non-circular symmetric shape. The first marker 41 and the second marker 42 may have a shape different from the annular shape (for example, a shape obtained by cutting an annular ring at any angle, a linear shape, or a coil shape obtained by spirally winding a wire).

For example, the arrangement of the distal tip 40, the first marker 41, and the second marker 42 can be arbitrarily changed. The first marker 41 does not need to overlap with the distal tip 40, and may be provided at a position adjacent to the proximal end of the distal tip 40 or a position away from the proximal end of the distal tip 40. The second marker 42 may be provided at a position different from the position adjacent to the distal end of the distal end opening 201 of the OTW tube 20 (for example, a position away from the distal end opening 201). The first marker 41 and the second marker 42 may be provided on a tube (the sensor tube 10 or the OTW tube 20) different from the RX tube 30. The first marker 41 and the second marker 42 may be provided on the same tube as described above, or may be provided on different tubes.

For example, at least one of the first outer tube 50 and the second outer tube 80 may be omitted. For example, in the example of the embodiment described above, each of the first outer tube 50 and the second outer tube 80 is formed of one layer, but at least one of the first outer tube 50 and the second outer tube 80 may be formed of two or more layers. For example, in the example of the embodiment described above, the transverse sectional shape of the portion of the catheter 1 covered with the first outer tube 50 is a triangular shape with round corners, but may be any shape such as a circular shape or an elliptical shape. For example, in the example of the embodiment described above, the transverse sectional shape of the portion of the catheter 1 covered with the second outer tube 80 is an elliptical shape, but may be any shape such as a circular shape or a triangular shape with round corners. For example, at least one of the first outer tube 50 and the second outer tube 80 may have an outer shape along the contour of the outer peripheral surfaces of the tubes 10 and 20 (or the tubes 10, 20, and 30).

For example, the shapes of the branching connector 60, the first reinforcing member 61 to the third reinforcing member 63, the cylindrical member 64, the connector 65, and the connector 25 described above are merely examples, and may be arbitrarily changed. For example, at least a part of the branching connector 60, the first reinforcing member 61, the second reinforcing member 62, and the cylindrical member 64 may be configured as a single member or may be omitted. For example, the third reinforcing member 63 and the connector 25 may be configured as one member. For example, the cylindrical member 64 may include a mechanism (e.g., a scale or a stopper provided for each prescribed length in the longitudinal direction, or a scale or a stopper provided for each prescribed angle in the circumferential direction) that assists adjustment of at least one of the front-back position of the sensor 70 and the orientation of the sensor 70 in the circumferential direction.

For example, the method for forming the coupling portion 95 described in the steps b1 to b8 is merely an example and can be variously changed. For example, in at least one of the steps b2 and b6, instead of immersing the tubes 10 and 30 in the liquid, the liquid of the base agent or the top agent may be sprayed to the tubes 10 and 30 to be applied to the surfaces. For example, in at least one of the steps b4 and b7, the control of the layer thickness by sucking up the liquid may be omitted.

### Modification 2

The configurations of the catheters 1 and 1A to 1E according to the first to sixth embodiments and the configurations of the catheters 1 and 1A to 1E according to the modification 1 may be appropriately combined. For example, the coupling portions 95C and 95D described in the fourth and fifth embodiments may be formed using the coating agent (one agent, three or more agents) described in the second and third embodiments. For example, in the configurations described in the second to fifth embodiments, the OTW tube 20 may be omitted according to the sixth embodiment.

### Aspects

(1) According to an aspect of the disclosed embodiments, a medical device is provided. The medical device includes a first tube having a first lumen and a second tube that has a second lumen and is coupled to the first tube with a coating agent in a part of a longitudinal direction of the medical device.

With this configuration, the first tube and the second tube can be coupled to each other with the coating agent without using heat fusion or an adhesive. For this reason, it is possible to suppress deformation of the first and second tubes, which occurs when the first and second tubes are couped to each other by heat fusion. In addition, it is possible to avoid disadvantages caused when coupling is provided using an adhesive, for example, a decrease in the flexibility and the operability due to curing of the coupling portion and a decrease in the acoustic characteristics in the coupling portion.
(2) In the medical device according to the above-described aspect, the first lumen may allow a sensor that acquires image information to be inserted to the first lumen. With this configuration, it is possible to configure the medical device as a device that can also use the sensor, which acquires image information.
(3) In the medical device according to the above-described aspect, the second tube may include an acoustic window in which a wall portion of the second tube is present over the entire circumferential direction, and coupling between the first tube and the second tube with the coating agent may be provided in at least a part of the acoustic window in the longitudinal direction. With this configuration, in at least a part of the acoustic window, in which sensing (acquisition of image information) by the sensor is performed, coupling is provided with a coating agent instead of an adhesive, and therefore, it is possible to prevent the sensing by the sensor from being inhibited by the adhesive.
(4) In the medical device according to the above-described aspect, in a transverse section of the medical device including a portion coupled with the coating agent, a space may be formed between a common external tangent line to the first tube and the second tube and outer peripheries of the first tube and the second tube. With this configuration, the sensing by the sensor can be performed more favorably by the space that does not hinder the sensing by the sensor.
(5) In the medical device according to the above-described aspect, the coating agent may be a hydrophilic coating base agent. With this configuration, the hydrophilic coating can play a role of coupling the first and second tubes.
(6) The medical device according to the above-described aspect may include a first marker having radiopacity and a second marker that has radiopacity and is provided closer to a proximal end side than the first marker, and a coupling portion between the first tube and the second tube with the coating agent may be provided in entirety between the first marker and the second marker. With this configuration, the entirety between the first marker and the second marker can be coupled with the coating agent.
(7) In the medical device according to the above-described aspect, the coupling portion between the first tube and the second tube with the coating agent may include a first coupling portion and a second coupling portion provided at a position away from the first coupling portion toward a proximal end side. With this configuration, the coupling portions of the first and second tubes can be provided at two locations away from each other.

Although the present mode has been described above based on the embodiments and the modifications, the embodiment of the above-described mode is intended to facilitate understanding of the present mode and does not limit the present mode. The present mode can be modified and improved without departing from the gist and the scope of the claims, and the present mode includes equivalents thereof. In addition, if the technical features are not described as essential in the present specification, the technical features can be appropriately deleted.

What is claimed is:

1. A medical device comprising:
a first tube having a first lumen; and
a second tube having a second lumen, wherein the first tube is coupled to the second tube by a coating layer along at least one coupling portion in a longitudinal direction of the medical device.

2. The medical device according to claim 1, wherein
the first lumen is configured to receive a sensor that acquires image information.

3. The medical device according to claim 1, wherein
a section of the medical device defines an acoustic window, the acoustic window corresponding to a region where a wall portion of the second tube extends over an entire circumferential direction thereof, and
the at least one coupling portion is provided in at least a part of the acoustic window in the longitudinal direction.

4. The medical device according to claim 1, wherein
in a transverse section of the medical device taken through the at least one coupling portion,
a space is formed between a common external tangent line to the first tube and the second tube and outer peripheries of the first tube and the second tube.

5. The medical device according to claim 1, wherein
the coating layer is made from a hydrophilic coating base agent.

6. The medical device according to claim 1, comprising:
a first marker having radiopacity; and
a second marker that has radiopacity and provided closer to a proximal end side than the first marker, wherein
the at least one coupling portion extends substantially entirely between the first marker and the second marker.

7. The medical device according to claim 1, wherein
the at least one coupling portion includes:
a first coupling portion; and
a second coupling portion provided at a position away from the first coupling portion toward a proximal end side.

8. The medical device according to claim 1, wherein the coating layer includes an undercoat layer applied to outer peripheral surfaces of the first tube and the second tube, the undercoat layer coupling the first tube and the second tube.

9. The medical device according to claim 8, wherein the coating layer further includes an overcoat layer applied over the undercoat layer.

10. The medical device according to claim 9, wherein the overcoat layer is hydrophilic.

11. A catheter, comprising:
an elongate catheter body having a proximal end and a distal end, the catheter body including:
a sensor tube defining a sensor lumen configured to receive an ultrasonic imaging sensor; and
a guidewire tube defining a guidewire lumen configured to slidably receive a guidewire, the guidewire tube being positioned adjacent to the sensor tube, wherein
the sensor tube and the guidewire tube are coupled to one another along a discrete coupling portion by a coating layer, the coating layer forming a thin film that adheres to outer surfaces of the sensor tube and the guidewire tube while providing a recess portion between the sensor tube and the guidewire tube.

12. A method for manufacturing a medical device, the method comprising:
placing a first tube having a first lumen adjacent to a second tube having a second lumen;
applying a liquid coating agent to at least a portion of outer surfaces of the adjacent first and second tubes; and
drying the liquid coating agent at a temperature below a melting point of the first tube and the second tube to form a solid coating layer that mechanically couples the first tube to the second tube.
